(19) United States
(12) Patent Application Publication
(10) Pub. No.: US 2024/0011987 A1
(43) Pub. Date: Jan. 11, 2024

(54) New Core/Shell Materials of Nanowire/Graphene on Low-Cost RFID Tags for Rapidly Sensing Live Cell Metabolites at Single-Cell Sensitivity

(71) Applicant: BOARD OF TRUSTEES OF THE UNIVERSITY OF ARKANSAS, Fayetteville, AR (US)

(72) Inventors: Zheng R. Tian, Fayetteville, AR (US); Chenguang Fan, Fayetteville, AR (US); Aseel Al-Baldaway, Fayetteville, AR (US); Rudy Acosta, Fayetteville, AR (US); Thomas McKean, III, Fayetteville, AR (US)

(73) Assignee: BOARD OF TRUSTEES OF THE UNIVERSITY OF ARKANSAS, Fayetteville, AR (US)

(21) Appl. No.: 18/471,276

(22) Filed: Sep. 20, 2023

Related U.S. Application Data
(63) Continuation of application No. 17/670,408, filed on Feb. 11, 2022.
(60) Provisional application No. 63/148,586, filed on Feb. 11, 2021.

(51) Int. Cl. G01N 33/569; G01N 33/543

(52) U.S. Cl. CPC G01N 33/56911; G01N 33/54313; G01N 2333/32; G01N 2333/245

(57) ABSTRACT

A biosensor having a core/shell nanocomposite of TiO2/rGO formed by hydrothermally coating reduced graphene oxide (rGO) flakes on titanate nanowires.

### RELATED APPLICATIONS

This application is a continuation of U.S. Ser. No. 17/670,408 filed on Feb. 11, 2022, which claims priority to U.S. Provisional Application No. 63/148,586, filed on Feb. 11, 2021, both of which are incorporated herein in their entirety.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Proton exchange membranes (PEM) have led to a new era of clean energy generated from hydrogen fuel. Reaching a multibillion-dollar market, PEM fuel cells (FC) have the potential to address the high usage of fossil fuels in the transportation and space industries. PEMFCs operate through an electrochemical process where hydrogen and oxygen reactants form electricity and the lone byproduct of water. Current configurations utilize a fluoropolymer with the trade name Nafion for its durability and long lifetime. Nafion conducts protons use an aqueous electrolyte and require extensive water management and lower operating temperatures. By increasing the operating temperature, not only will the infrastructure necessary for water management no longer be required, lowering the device's footprint, as much as a 10% cost decrease is estimated to be possible through an increase in reaction kinetics, increased fuel impurity tolerance, and use of less expensive catalyst materials.

Somewhere between 100-200° C. is thought to be the optimal operating temperature for PEMFCs, since increasing the temperature much more comes with significant cost considerations. One strategy to create a PEM that operates within this range is to use the polymer polybenzimidazole (PBI), known for its thermal and mechanical durability in applications such as space suits and fire protection clothing. PBI is capable of housing a strong acid electrolyte that preserves the efficient proton conductivity of the water electrolyte at temperatures beyond what water is capable of operating at. The problem with this configuration is the corrosive nature of the phosphoric acid electrolyte and its impact on the durability and lifetimes of the PBI membrane. For practical use, the durability of these PEMs must be improved.

A common method to increase the durability of polymer membranes is to integrate a nanomaterial filler into the membrane to form a polymer nanomaterial composite. The nanomaterial increases both the mechanical stability and the proton conductivity of the resulting composite membranes by stabilizing the polymer chains and providing more active surface area, respectfully. The most effective composite should contain a high amount of nanomaterial uniformly dispersed throughout the membrane using a material known for strong proton conductivity. Both criteria possess inherent issues that limit their applicability. First, when trying to incorporate a high amount of nanomaterial into a membrane casted using traditional methods, the presence of the nanomaterial causes the viscosity of the precursor solution to increase drastically to a point where it quickly becomes unusable. Additionally, even at low concentrations, the nanomaterial tends to agglomerate and resist evenly dispersing throughout. Second, proton conductivity in nanomaterials is typically reliant on oxygen vacancies present in the material's structure. These vacancies are formed using a process known as sintering, where the material is exposed to a high-powered laser at high temperature and pressure. At a large scale, these conditions would impose tremendous cost and would likely serve as a process bottleneck.

To decrease the cost and footprint of PEMFCs and utilize the great potential of clean hydrogen energy, the operating temperature of the devices must increase. A PBI nanomaterial composite membrane will allow for strong performance at elevated temperatures, but current PBI membranes are not durable enough for practical application and ideal composite membranes that may show practical levels of performance and durability do not yet exist. Therefore, a need for both a low-cost and proton-conductive nanomaterial and a durable composite PBI membrane containing a uniformly dispersed amount of said nanomaterial exists.

### BRIEF SUMMARY OF THE INVENTION

In one embodiment, the present invention provides a biosensor having a core/shell nanocomposite of TiO2/rGO formed by hydrothermally coating reduced graphene oxide (rGO) flakes on titanate nanowires.

In other embodiments, the present invention provides a nanocomposite-modified RFID tag that has been proven to be a new type of biosensor with electrochemical impedance in the frequency range of 730-930 MHz.

In other embodiments, the present invention provides a biosensor that can detect both Gram-negative and Gram-positive bacteria E. coli, S. LT2, and B. subtilis, respectively in real-time, each with the detection limit in the single-cell level. The low detection limit and small quantity of samples are better than that of other methods for detecting live bacteria in literature.

In other embodiments, the present invention provides a biosensor that can detect live bacteria in both foods- and nonfood-products in industrial sectors, which can help largely reduce the annual cost of medical treatment, lost productivity, and illness-related mortality at $55.5 billion from the bacteria infection.

In other embodiments, the present invention provides a biosensor that has increase selectivity by functionalizing the core/shell surface with biomarkers (such as antibodies and aptamers) and other nanoparticles to selectively detect the target directly.

In other embodiments, the present invention provides a biosensor that improves the physical contact between the nanowires and the RFID tag, using a conducting polymer-based glue.

In other embodiments, the present invention provides a biosensor that directly monitors for the presence of pathogenic bacteria.

In other embodiments, the present invention provides a biosensor that detects viruses or other microorganisms.

In other embodiments, the present invention concerns PBI composite membranes containing a high content of a low-cost and proton-conductive nanomaterial filler. In this embodiment, the PBI and nanomaterial share similar surface chemistries and the nanomaterial incorporated into the polymer solution at a much higher content without causing the solution to become too viscous to cast. By first subjecting the nanomaterial to a process where a PBI coating is formed on its surface to form a “bridge layer”, the nanomaterial efficiently disperses through the network of polymer chains. This 2-step process (form bridge layer, then cast) was tested using different precursor solution configurations to produce PBI composite PEMs with varying nanomaterial content. By creating this process, a potential avenue to creating PBI PEMs with enough nanomaterial filler to reach levels of performance and durability suitable for application was created, which provides progress toward providing the field of PEMFCs a device with a higher operating temperature than current state of the art.

It is to be understood that both the foregoing general description and the following detailed description are exemplary and explanatory only and are not restrictive of the invention, as claimed.

explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1A is an SEM of the nanowires that may be used with an embodiment of the present invention.

FIG. 1B shows intensity versus to 2 Theta-Theta for Titanate and rGO.

FIG. 1C is a schematic of an embodiment of the present invention involving a biosensor.

FIG. 1D is an SEM of showing how the embodiment shown in FIG. 1C detects Bacillus subtilis.

FIG. 1E is a detection system for an embodiment of the present invention.

FIG. 1F shows testing results of modified and non-modified tags.

FIG. 2 shows detection of detection of bacterial metabolic products using the RFID tags of the present invention various types of bacteria such as E. coli MG1655, Salmonella LT2, and Bacillus subtilis.

FIG. 3 is a visual summary of the casting of high filler content membranes. The membranes darkened as they progressed from the pure PBI at the bottom to the most concentrated at the top (right). From top the bottom the membranes contain nanomaterial contents of 50%, 25%, 10%, 0.5%, and Pure PBI.

FIG. 4 show an average proton conductivity of each membrane class by temperature.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention.

In certain embodiments, the present invention provides a biosensor comprising a titanate/rGO core/shell nanowires and radiofrequency technique for real-time detection of bacterial metabolic products using RFID tags with three types of bacteria: E. coli MG1655, Salmonella LT2, and Bacillus subtilis.

Graphene oxide (GO) was prepared using a modified Hummer's method. Briefly 32, 23 mL of concentrated sulfuric acid (18M) was stirred in an ice bath for 15 minutes before adding 0.5 g of graphite powder and 0.5 g of sodium nitrate. The resulting mixture was stirred for another 15 minutes. Then, 4.0 g of potassium permanganate was added very slowly. The mixture was stirred for an additional 30 minutes then transferred to a 40° C. water bath, stirred for 90 minutes, and then slowly transferred into 50 mL of deionized (DI) water with stirring, followed by the dropwise addition of 6 mL of 30% hydrogen peroxide, forming a golden-brown mixture. The mixture was diluted by another 100 mL of DI water. The final mixture was washed, via centrifugation using centrifuge tubes (each 50 mL), with HCl (5 wt %), then acetone (twice), and finally with DI water repeatedly until pH near 7 to obtain the GO.

Titanate/rGO core-shell nanowires were synthesized using a hydrothermal process. In a typical preparation, 0.1 g GO was added to 40 mL of DI water and exfoliated in an ultrasonic bath for 10 minutes. Then, 1 g of titanium dioxide powder and 16 g of sodium hydroxide pellets were added to the solution. The final solution was stirred for 5 minutes and sonicated for 15 minutes before being transferred to a Teflon lined autoclave which was heated to 240° C. for 72 hours.

Morphologies of titanate/rGO nanowires were observed by an optical microscope (Olympus BX41), a scanning electron microscope (SEM, Tescan VEGA II SBH), and TEM (FEI Tecnai G2 F20 S-Twin microscope). The samples were characterized using X-ray Diffractometer (XRD, Rigaku MiniFlex II).

Salmonella LT2 strain and E. coli MG1655 strain were inoculated into 2 mL of fresh LB media from glycerol stock and grown at 37° C. overnight. On the second day, 2 μL overnight culture was inoculated into 2 ml fresh LB media and grown at 37° C. for 12 hours. Cells were pelleted by centrifugation at 5,000×g for 1 minute. Bacillus subtilis was cultured in 10 mL of nutrient broth media and incubated at 37° C. for 12 hours. Then, cells were pelleted by centrifugation at 5,000×g for 5 minutes. The growth of bacteria was evaluated using plate count (3M petrifilm plate).

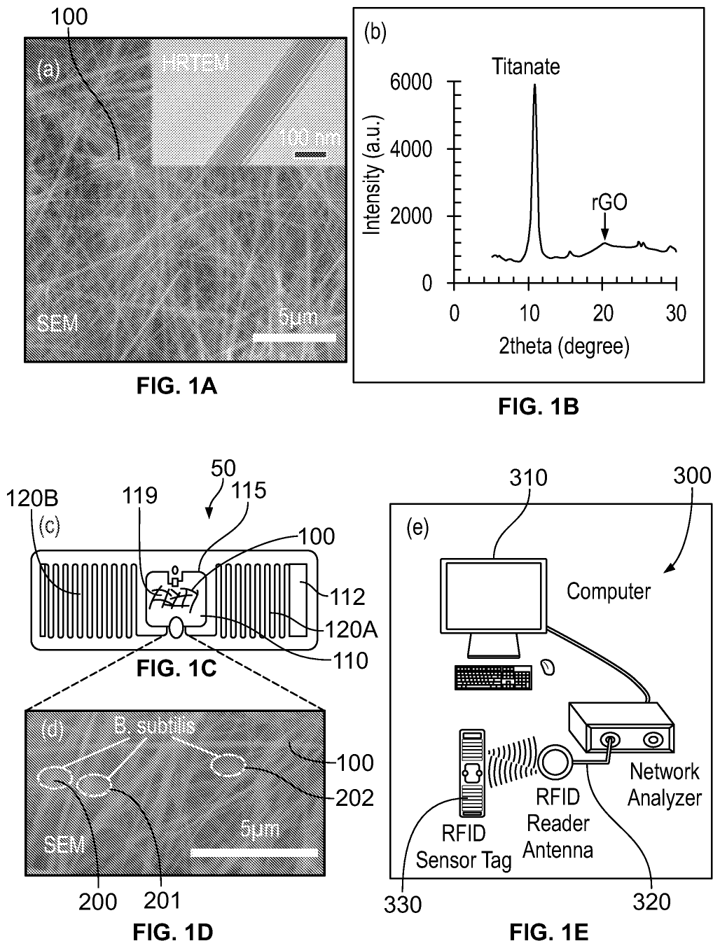

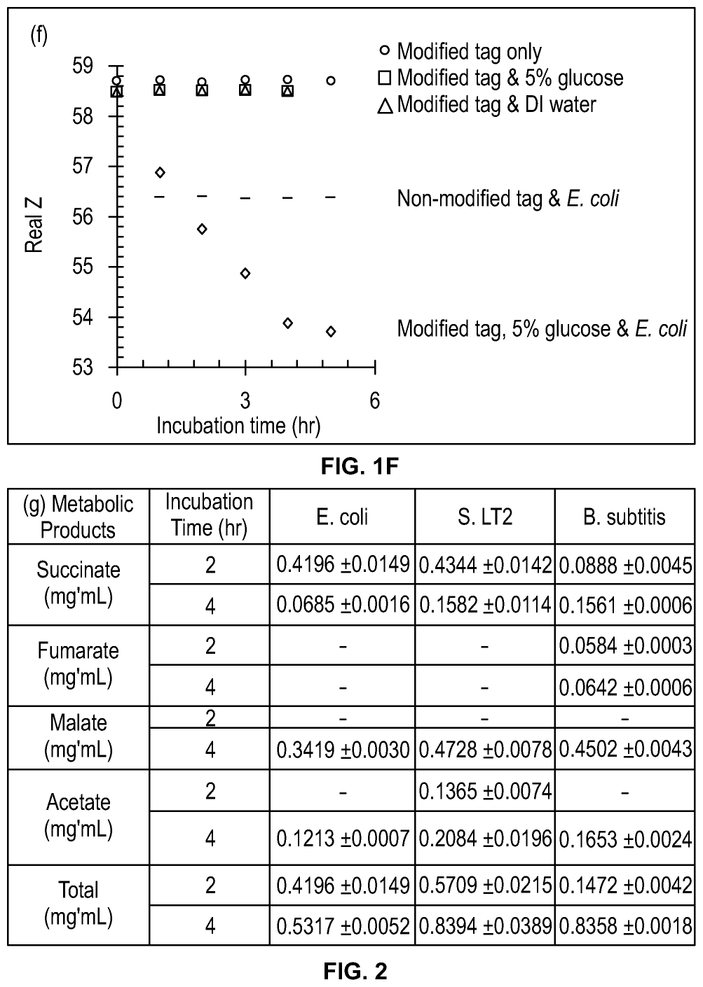

In other embodiments, as shown in FIG. 1A-1D, the present invention provides a biosensor 50 having nanowires 100 which are wrapped with reduced graphene oxide into the TiO2/rGO type of core/shell nanostructure. Nanowires 100 are coated on the middle part 110 of tag 112 where the two sides of the antenna 120A and 120B of the tag meet to form a detection zone 119 on RFID tag 112 surface 115. Detection zone 119 is capable of detecting single live bacterium's metabolic byproducts that changed the RFID tag's impedance in real-time. As shown in FIG. 1D, which is an SEM of biosensor 50, this embodiment of the present invention is capable of detecting Bacillus subtilis 200-202 as a result of biosensor 50 having an electrochemical impedance in the frequency range of 730-930 MHz.

In other aspects, the present invention concerns a detection system 300 comprised of a computer 310, RFID reader 320 and an RFID sensor tag 330 which may be in the form as described herein. Also, detection zone 119 may further include biomarkers (such as antibodies and aptamers) and other nanoparticles to selectively detect the target directly.

Passive RFID tag 112 (Alien, ALN-9610) was prepared after cleaning the aluminum surface from plastic layers and the glue between the layers with ethanol. A drop of titanate/rGO core/shell nanowires 100 (0.03 mg\ml) was added on the middle part of tag 112 where the two sides of the antenna of the tag meet, and it was left under 37° C. for 15 minutes. For preparing bacteria in glucose medium, glucose solution has been prepared in different concentrations (0.2, 1, 2.5, 5) % in water. They were filtered through a 0.2 μm filter for sterilizing purposes. Then, different concentrations of bacterial cells (102, 103 CFU/ml) were prepared using glucose medium. Afterward, testing the modified tag was performed via a vector network analyzer under computer control. To scan the range of interest frequencies and collect the response of the impedance from the RFID sensor, the network analyzer (DG8SAQ VNWA 3E, SDR-Kits) was used. All the collected signals and impedance data have been analyzed using DG8SAQ Vector network analyzer software Ver. 36.7.6. After setting the network analyzer, the modified tag was fixed on the antenna loop. Then, 10 μl of the bacteria dilutions were exposed to the part that was coated with the nanowire of the tag at various numbers of bacterial cells (0.5×102, 102, 103 CFU/ml) and glucose concentrations (0.2, 1, 2.5, 5%). Real Z was taken before and after exposure on an hourly basis until cell germination stalled. The tag has been used many times after wiping the tag with ethanol.

Samples of different dilutions (102, 103) CFU\ml of the three types of bacteria in different concentrations (0.2, 1, 2.5, 5) % of glucose were measured every hour until pH values started. 15 ml from each sample was taken for measurements. All the experiment steps occur at room temperature.

The organic acids in every sample were quantified using an HPLC system. The bacterial growth media and different organic acids including citric acid, formic acid, acetic acid, fumaric acid, lactic acid, succinic acid, and malic acid as standards were analyzed with the HPLC (Milford, Mass., U.S.A.) and Refractive Index (HPSEC-RI) detector. The standards were prepared in different concentrations (1, 2, 3, 4, 5 mg/mL). 20 mL of 107 CFU/mL of each type of the three bacteria in the study were prepared in 2.5% glucose. Each of the bacterial suspension was taken into a tube and incubated for 0, 2, and 4 hours. Then, all the samples were filtered two times through the sterile syringe filter (0.2 μm cellulose acetate, Watman) for sterilization. Samples (50 μL) were injected into a Waters HPLC system, with an HPLC isocratic pump (Model 1515), and a manual injector. The elution was performed by an isocratic flow of 0.025 (M) H2SO4 as a mobile phase with a flow rate of 0.4 mL/min through a C18 column (Phenomenex Rezex ROA-Organic Acids 15×7.8 mm). Eluted compounds were detected by a Waters index reflective detector (Model 2414) which was set at 40° C. The column was maintained at ° C. in a column heater. Organic acids were quantified using 5-point calibration curves with standards.

In other embodiments, the present invention concerns a process to create PBI composite membranes filled with a high content of nanomaterial filler. PBI membranes are traditionally cast by first forming a precursor solution using a compatible solvent, typically dimethylacetamide. This solution is evenly spread over a glass surface then placed in a vacuum oven to force the solvent to evaporate and allow the membrane to form. To form a composite membrane, the nanomaterial is added to the precursor solution and is integrated into the membrane during casting. The problem arises when a larger amount of nanomaterial is added to the precursor solution and the solution becomes too viscous to evenly spread over the glass surface. This increase in viscosity is a result of the difficulty of the long polymer chains to smoothly move in the solution when the nanomaterial is introduced. Since the polymer and nanomaterial are incompatible, they tend to cluster together into separate phases that impede each other's movement. To overcome this issue, a nanomaterial that is compatible with the polymer must be chosen.

PBI has a structure made up of mostly carbon rings, and these can interact with other carbon rings. Bonds between carbons in carbon rings do not use all the available orbitals when forming, and these leftover orbitals can interact with leftover orbitals of other carbon rings in what is known as 1T-1T interactions.

By using graphene oxide as the major component of the chosen nanomaterial filler, a proton conductive reduced graphene oxide/titanate nanowire composite (RGONF) if formed where the carbon rings present in the graphene oxide interact with the carbon rings in the PBI structure. Testing shows that these 1T-1T interactions are strong enough to form a persistent layer of PBI on the surface of the composite nanomaterial. This “bridge layer” allows the nanomaterial to integrate with the PBI chains and form a much more uniform precursor solution. This precursor solution can be evenly spread on the surface of a glass slide as if it did not have a large amount of nanomaterial. This process allows for the formation of a membrane with a novel polymer/nanomaterial configuration provides a durable PBI PEM capable of addressing the commercial need for a higher operating temperature PEM.

Typical PBI composite PEMs do not show nearly as high nanomaterial amount. The embodiments of the present invention use a nanomaterial that contains a carbon ring structure capable of interacting with the PBI's carbon rings. This process uses the RGONF as the carbon ring-containing filler. The RGONF was chosen for its low cost and ease of introducing proton conductivity. The material is synthesized through a cost-effective hydrothermal process, and by performing a simple ion exchange to introduce hydrogen ions the material becomes proton conductive. First, the protonated RGONF (synthesized) is coated by adding it to a thin PBI solution, approximately 4% polymer (obtained from PBI Performance Products inc.) and stirring until dissolved (approximately 2 hours). Once dissolved the solution is sonicated for 10 minutes to ensure the material is sufficiently coated then centrifuged to isolate the now coated material. The supernatant is discarded, and the nanomaterial paste leftover is added to a thicker PBI solution (approximately 15%, obtained from PBI Performance Products inc.). The goal of this addition is to create a solution with the maximum amount of nanomaterial paste and minimum amount of PBI solution. First a very small amount of PBI solution is added to an empty vial, just enough to coat the bottom of the vial and prevent the nanomaterial from touching the glass when added. Then, the nanomaterial paste is added to this small volume of PBI solution under stirring. Next, more PBI solution is added to the solution until a sufficient volume of solution is present to coat the glass surface. Finally, this solution is stirred until uniform and then spread evenly on the glass surface. At this point the membrane is dried in the vacuum oven and processed identically to a pure PBI or low nanomaterial content membrane.

To confirm the successful formation of the PBI “bridge layer”, the nanomaterial was examined with Fourier Transform Infrared Spectroscopy (FTIR). PBI is characterized by the presence of an N—H bond, which can be observed at 3400 wavenumbers using FTIR. The composite nanomaterial was subjected to the coating process then placed under the FTIR. The results indicated the presence of the N—H bond and therefore the polymer. Next, the process was carried out with three different precursor solutions with different compositions. Each solution yielded a successful membrane, and the resulting membranes differed both from each other and greatly from a pure polymer membrane.

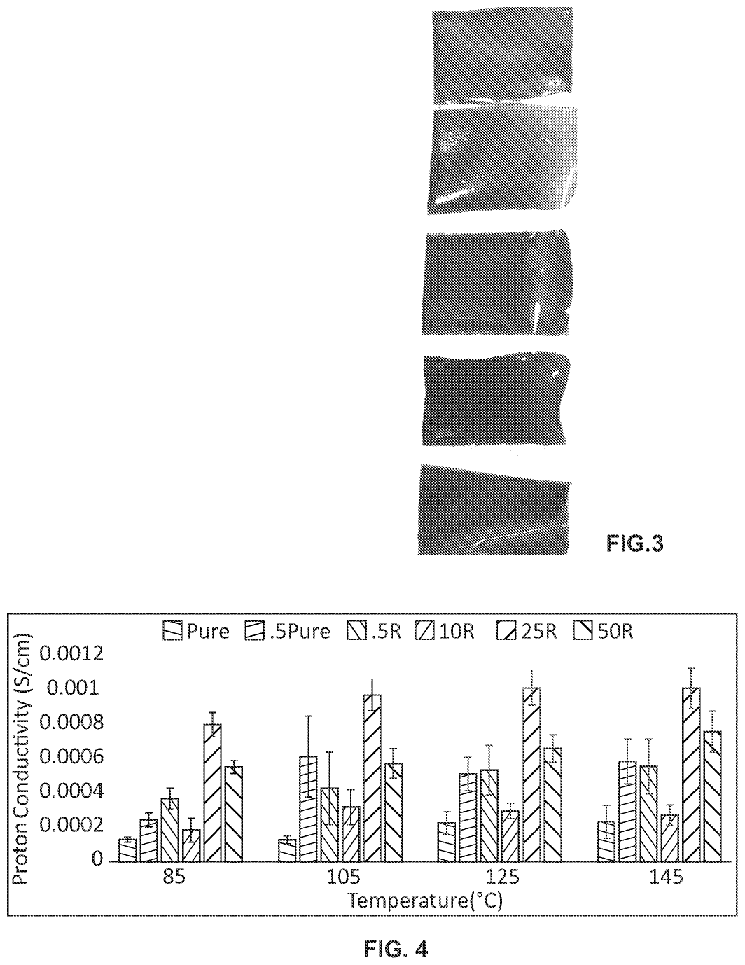

FIG. 3 is a visual summary of the casting of high filler content membranes. The membranes darkened as they progressed from the pure PBI at the bottom to the most concentrated at the top (right). From top the bottom the membranes contain nanomaterial contents of 50%, 25%, 10%, 0.5%, and Pure PBI. FIG. 4 show an average proton conductivity of each membrane class by temperature.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A method to perform real-time detection of bacterial metabolic products comprising the steps of: providing a sensor comprising: an RFID tag, said RFID tag having a centrally located nanocomposite coating; said coating comprised of nanowires wrapped with reduced graphene oxide into the TiO2/rGO type of core/shell nanostructure; said coating forming a detection zone causing said RFID tag to have an electrochemical impedance that changes when in contact with a bacterial metabolic product; and placing a substance to be tested on said detection zone.

2. The method of claim 1 wherein said core/shell nanocomposite of TiO2/rGO is formed by hydrothermally coating reduced graphene oxide (rGO) flakes on titanate nanowires.

3. The method of claim 1 wherein said RFID tag has an electrochemical impedance in the frequency range of 730-930 MHz.

4. The method of claim 1 wherein said sensor is adapted to detect both Gram-negative and Gram-positive bacteria E. coli, S. LT2, and B. subtilis, respectively.

5. The method of claim 1 wherein said sensor is adapted to have a detection limit in the single-cell level.

6. The method of claim 1 wherein said detection is adapted to detect a single live bacterium's metabolic byproducts.

7. The method of claim 1 wherein said detection zone further includes biomarkers (such as antibodies and aptamers) and other nanoparticles to selectively detect the target directly.

8. The method of claim 1 wherein said detection is adapted to detect the presence of pathogenic bacteria.

9. A method to perform real-time detection of a virus comprising the steps of: providing a sensor comprising: an RFID tag, said RFID tag having a centrally located nanocomposite coating; said coating comprised of nanowires wrapped with reduced graphene oxide into the TiO2/rGO type of core/shell nanostructure; said coating forming a detection zone causing said RFID tag to have an electrochemical impedance that changes when in contact with a virus; and placing a substance to be tested on said detection zone.

10. The method of claim 9 wherein said core/shell nanocomposite of TiO2/rGO is formed by hydrothermally coating reduced graphene oxide (rGO) flakes on titanate nanowires.

11. The method of claim 9 wherein said RFID tag has an electrochemical impedance in the frequency range of 730-930 MHz.

12. The method of claim 1 wherein said detection zone further includes biomarkers (such as antibodies and aptamers) and other nanoparticles to selectively detect the target directly.

13. A method to perform real-time detection of microorganisms comprising the steps of: providing a sensor comprising: an RFID tag, said RFID tag having a centrally located nanocomposite coating; said coating comprised of nanowires wrapped with reduced graphene oxide into the TiO2/rGO type of core/shell nanostructure; said coating forming a detection zone causing said RFID tag to have an electrochemical impedance that changes when in contact with a microorganism; and placing a substance to be tested on said detection zone.

14. The method of claim 13 wherein said core/shell nanocomposite of TiO2/rGO is formed by hydrothermally coating reduced graphene oxide (rGO) flakes on titanate nanowires.

15. The method of claim 13 wherein said RFID tag has an electrochemical impedance in the frequency range of 730-930 MHz.

16. The method of claim 13 wherein said detection zone further includes biomarkers (such as antibodies and aptamers) and other nanoparticles to selectively detect the target directly.
